(19) United States
(12) Patent Application Publication
(10) Pub. No.: US 2007/0168222 A1
(43) Pub. Date: Jul. 19, 2007

(54) System and method for providing hierarchical medical device control for automated patient management

(76) Inventors: Kenneth P. Hoyme, Plymouth, MN (US); Howard D. Simms, Shoreview, MN (US); Alan H. Smythe, White Bear Lake, MN (US)

Correspondence Address: CASCADIA INTELLECTUAL PROPERTY, 500 UNION STREET, STE.1005, SEATTLE, WA, 98101, US

(21) Appl. No.: 11/336,736

(22) Filed: Jan. 19, 2006

(51) Int. Cl. G06Q 10/00; A61B 5/00

(52) U.S. Cl. 705/2; 600/300

(57) ABSTRACT

A system and method for providing hierarchical medical device control for automated patient management is presented. A processor is operatively coupled to a plurality of medical devices on a substantially continual basis to receive sensor data. A control strategy is assigned to the processor to specify actions to be taken by the medical devices to affect the attainment of a therapy goal. State is maintained, selected from the group comprising a history of changes to the control strategy and past sensor data received from the medical devices. Feedback is periodically received. The feedback includes new sensor data from the medical devices. The feedback and the state are analyzed against the actions specified in the control strategy. Control is provided to one or more medical device in response to an actionable change from the actions specified in the control strategy.

### FIELD OF THE INVENTION

The present invention relates in general to automated patient management and, specifically, to a system and method for providing hierarchical medical device control for automated patient management.

### BACKGROUND OF THE INVENTION

In general, implantable medical devices (IMDs) can provide in situ therapy or monitoring under preprogrammed autonomous control. Autonomous control is governed by tunable and fixed control parameters, which are physician-selected to meet therapy goals. IMDs must be periodically interfaced to external devices, such as programmers and patient management devices, for physician follow-up. Physicians assess a patient's current condition based on downloaded patient data and lab or clinical tests, such as electrophysiology tests, treadmill stress tests, and blood work, to determine if treatment goals are being met or whether control parameters require reprogramming.

IMD therapy is intended to meet specific goals and therapy is selected based upon physician experience and population data for comparable patient outcomes. A therapy goal is implemented by specifying actions to be performed by the IMD under a treatment plan through downloadable control parameters. The medical means for implementing a treatment plan will depend upon the patient profile and medical resources available.

Progress towards a therapy goal can be gauged in light of the scope of control over and the therapeutic affect made upon a patient. For example, an IMD exercises direct control over a patient. IMD resources are constrained in terms of processing, storage, and power budget. As a result, IMDs can only provide a temporally limited perspective of the efficacy of the therapy provided due to the restricted memory available.

Implementing goal-directed operation on IMDs can be a challenge. Therapy is directed to patient management at a micro level and IMDs lack the resources to maintain and track progress towards a goal defined more broadly than an event occurrence. Goal-directed patient management is better handled at a macro level as provided on an external device, such as a server or patient management device. External devices allow patient data to be downloaded and tracked over time to build a more comprehensive picture of the patient's progress and therapy can be adjusted as necessary. Conventional approaches to goal-directed patient management, however, adopt open loop control strategies that require the involvement of a clinician, such as a physician, nurse, or other qualified individual.

U.S. Pat. No. 6,416,471, issued to Kumar et al. (“Kumar”) discloses a remote patient telemonitoring device. A disposable sensor band with electrode patches detects and transmits vital signs data to a signal transfer unit, which can either be worn or be positioned nearby the patient. The base station receives data transmissions from the signal transfer unit for transferring the collected data to a remote monitoring station. Indications are provided to a patient from the base station when threshold violations occur, but the system requires an operator, such as a physician or nurse, to manually review and provide an interpretation of the patient data.

U.S. Pat. No. 6,024,699, issued to Surwit et al. (“Surwit”) discloses a central data processing system configured to communicate with and receive data from a plurality of patient monitoring systems, which may implement a medical dosage algorithm to generate dosage recommendations. Blood from a pricked finger may be read on a chemically treated strip. Modifications to medicine dosages, medicine dosage algorithms, patient fixed or contingent self-monitoring schedules, as well as other treatment information are communicated, but screen mechanisms are provided to case managers for ensuring that treatment or information provided is medically sound before communicating that treatment or information to the patient or patient management device.

U.S. Pat. No. 6, 083,248, issued to Thompson discloses a worldwide patient location and data telemetry system for implantable medical devices. An implanted device telemetry transceiver within the implanted medical device communicates data and operating instructions to and from a medical device in a coded communication. A global positioning system provides data identifying the position of the patient. Should a need to upgrade or change the behavior of implanted devices arise, the system allows the central monitoring site to revise interfaced IMDs by transmitting new programming instructions, assuming appropriate governmental authorities and patients′physicians have agreed to the need for such changes.

Therefore, there is a need for providing remote patient management with closed loop reporting and control in a hierarchical structure that allows goal and sub-goal delegation and therapy feedback through levels of distributed control.

### SUMMARY OF THE INVENTION

A system and method includes a closed loop control hierarchy having two or more levels. The top, or root level, contains a server that centrally manages a control strategy. The penultimate level includes medical devices, such as implantable or external medical devices or sensors, and the bottom, or terminal, level includes their respective sensors and effectors. In one embodiment, the control hierarchy includes an intermediate logical level that includes one or more patient management devices, each dedicated to servicing one or more of the medical devices. Both the server and each patient management device function as controllers over physical plants that can include those devices assigned to children nodes in the control hierarchy. Control is exercised over and feedback is received from the assigned devices. Control is exercised by delegating sub-goals to the assigned devices. Feedback is analyzed against locally-maintained state to identify whether changes to the existing control strategy are necessary.

One embodiment provides a system and method for providing hierarchical medical device control for automated patient management. A processor is operatively coupled to a plurality of medical devices on a substantially continual basis to receive sensor data. A control strategy is assigned to the processor to specify actions to be taken by the medical devices to affect the attainment of a therapy goal. State is maintained, selected from the group comprising a history of changes to the control strategy and past sensor data received from the medical devices. Feedback is periodically received. The feedback includes new sensor data from the medical devices. The feedback and the state are analyzed against the actions specified in the control strategy. Control is provided to one or more medical device in response to an actionable change from the actions specified in the control strategy.

Still other embodiments of the present invention will become readily apparent to those skilled in the art from the following detailed description, wherein are described embodiments of the invention by way of illustrating the best mode contemplated for carrying out the invention. As will be realized, the invention is capable of other and different embodiments and its several details are capable of modifications in various obvious respects, all without departing from the spirit and the scope of the present invention. Accordingly, the drawings and detailed description are to be regarded as illustrative in nature and not as restrictive.

### BRIEF DESCRIPTION OF THE DRAWINGS

FIG. 1 is a functional block diagram showing, by way of example, an automated patient management environment.

FIG. 2 is a tree diagram showing, by way of example, a control hierarchy of medical devices for use in the patient management environment of FIG. 1.

FIG. 3 is a data flow diagram showing hierarchical medical device control and feedback in the patient management environment of FIG. 1.

FIGS. 4A-C are Venn diagrams showing, by way of example, data sets as maintained in the patient management environment of FIG. 1.

FIG. 5 is a graph showing the relationship between sampling frequency and sample size.

FIG. 6 is a flow diagram showing a method for providing hierarchical medical device control for automated patient management for use on the server of FIG. 3.

FIG. 7 is a flow diagram showing a method for providing hierarchical medical device control for automated patient management for use on a patient management device of FIG. 3.

FIG. 8 is a flow diagram showing a method for providing hierarchical medical device control for automated patient management for use on a medical device of FIG. 3.

FIG. 9 is a block diagram showing a system for providing hierarchical medical device control for automated patient management for use on the server of FIG. 3.

FIG. 10 is a block diagram showing a system for providing hierarchical medical device control for automated patient management for use on a patient management device of FIG. 3.

FIG. 11 is a block diagram showing a system for providing hierarchical medical device control for automated patient management for use on a medical device of FIG. 3.

### DETAILED DESCRIPTION

Automated Patient Management Environment

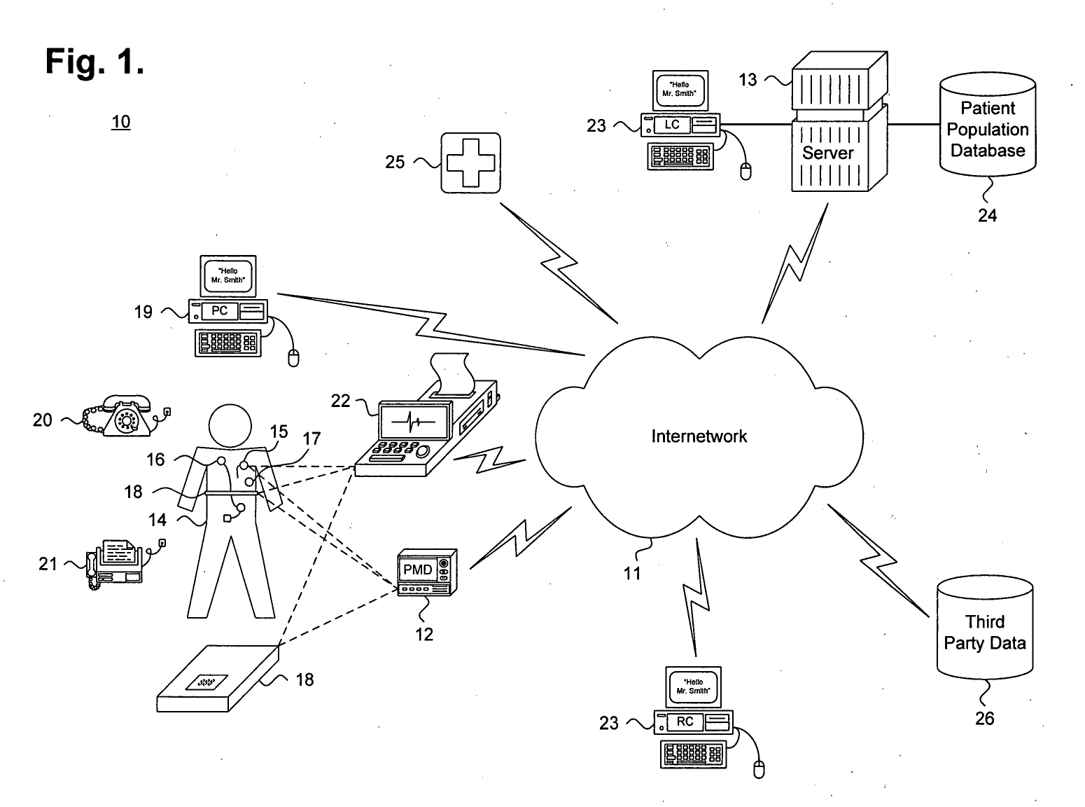

Automated patient management encompasses a range of activities, including remote patient management and automatic diagnosis of patient health, such as described in commonly-assigned U.S. Pat. application Pub. No. US2004/0103001, published May 27, 2004, pending, the disclosure of which is incorporated by reference. Such activities can be performed proximal to a patient, such as in the patient's home or office, centrally through a centralized server, such from a hospital, clinic or physician's office, or through a remote workstation, such as a secure wireless mobile computing device. FIG. 1 is a functional block diagram showing, by way of example, an automated patient management environment 10. In one embodiment, a patient 14 is proximal to one or more patient monitoring or communications devices, which are interconnected remotely to a centralized server 13 over an internetwork 11, such as the Internet, or through a public telephone exchange (not shown), such as a conventional or mobile telephone network. The patient monitoring or communications devices non-exclusively include a patient management device 12, such as a repeater, personal computer 19, including a secure wireless mobile computing device, telephone 20, including a conventional or mobile telephone, and facsimile machine 21. In a further embodiment, a programmer 22, such as a programmer or programmer-recorder monitor, can be used by clinicians, such as physicians, nurses, or qualified medical specialists, to interrogate and program medical devices. Finally, the centralized server 13 is remotely interfaced to a patient care facility 25, such as a clinic or hospital, to ensure access to medical response or patient care providers. Other patient monitoring or communications devices are possible. In addition, the internetwork 11 can provide both conventional wired and wireless interconnectivity. In one embodiment, the internetwork 11 is based on the Transmission Control Protocol/Internet Protocol (TCP/IP) network communication specification, although other types or combination of networking implementations are possible. Similarly, other network topologies and arrangements are possible.

Each patient management device 12 is uniquely assigned to a patient under treatment 14 to provide a localized and network-accessible interface to one or more medical devices, which serve as patient data sources 15-18, either through direct means, such as wired connectivity, or through indirect means, such as inductive coupled telemetry, optical telemetry, or selective radio frequency or wireless telemetry based on, for example, “strong” Bluetooth or IEEE 802.11 wireless fidelity “WiFi” and “WiMax” interfacing standards. Other configurations and combinations of patient data source interfacing are possible.

Patient data includes physiological measures, which can be quantitative or qualitative, parametric data regarding the status and operational characteristics of the patient data source itself, and environmental parameters, such as the temperature, barometric pressures, or time of day. The patient data sources collect and forward the patient data either as a primary or supplemental function. Patient data sources 15-18 include, by way of example, medical devices that deliver or provide therapy to the patient 14, sensors that sense physiological data in relation to the patient 14, and measurement devices that measure environmental parameters occurring independent of the patient 14. Other types of patient data are possible, such as third party data 26 received from external data sources, including repositories of empirical studies, public and private medical databases, patient registries, and the like. Additionally, current clinician-established guidelines associated with treatment can help to guide acceptable best practice treatment for patient care. Each patient data source can generate one or more types of patient data and can incorporate one or more components for delivering therapy, sensing physiological data, measuring environmental parameters, or a combination of functionality.

In a further embodiment, data values can be entered by a patient 14 directly into a patient data source. For example, answers to health questions could be input into a measurement device that includes interactive user interfacing means, such as a keyboard and display or microphone and speaker. Such patient-provided data values could also be collected as patient information. Additionally, measurement devices are frequently incorporated into medical therapy devices and medical sensors. Medical therapy devices include implantable medical devices (IMDs) 15, such as pacemakers, implantable cardiac defibrillators (ICDs), drug pumps, and neuro-stimulators, and external medical devices (EMDs) 16, such as automatic external defibrillators (AEDs). Medical sensors include implantable sensors 17, such as implantable heart and respiratory monitors and implantable diagnostic multi-sensor non-therapeutic devices, and external sensors 18, such as 24-hour Holter arrhythmia monitors, ECG monitors, weight scales, glucose monitors, oxygen monitors, and blood pressure monitors. Other types of medical therapy, medical sensing, and measuring devices, both implantable and external, are possible.

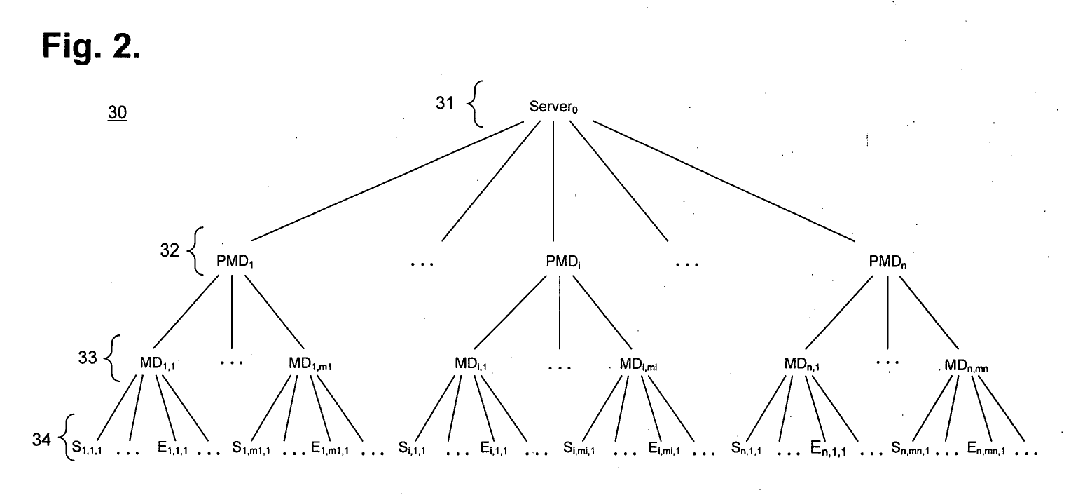

The patient management device 12 collects and temporarily stores patient data from the patient data sources 15-18 for periodic upload over the internetwork 11 to the server 13 and storage in a patient population database 24. Each patient 14 can be remotely managed through hierarchical control exercised by the server 13 over the patient management devices 12 and patient data sources 15-18, as further described below beginning with reference to FIG. 2. Briefly, a clinician defines a therapy goal for a patient based on a stored physiological assessment of a diagnosed disease state. The server 13 defines a control strategy for meeting a therapy goal and the control strategy is delegated to each of the devices through goals and sub-goals to form a closed loop control system. Control flows downward through the hierarchy, increasing in specificity with each decreasing level, and feedback flows upward, increasing in detail and temporal scope with each increasing level.

Each patient data source 15-18 collects the quantitative physiological measures on a substantially continuous basis and also records the occurrence of events, such as therapy or irregular readings. In a still further embodiment, the patient management device 12, personal computer 19, telephone 20, or facsimile machine 21 record or communicate qualitative quality of life (QOL) measures that reflect the subjective impression of physical well-being perceived by the patient 14 at a particular time. Other types of patient data collection, periodicity and storage are possible.

In a further embodiment, the collected patient data can also be accessed and analyzed by one or more clients 23, either locally-configured or remotely-interconnected over the intemetwork 11. The clients 23 can be used, for example, by clinicians to securely access stored patient data assembled in the database 24 and to select and prioritize patients for health care provisioning, such as respectively described in commonly-assigned U.S. patent application, Ser. No. 11/121,593, filed May 3, 2005, pending, and U.S. patent application, Ser. No. 11/121,594, filed May 3, 2005, pending, the disclosures of which are incorporated by reference. Although described herein with reference to physicians or clinicians, the entire discussion applies equally to organizations, including hospitals, clinics, and laboratories, and other individuals or interests, such as researchers, scientists, universities, and governmental agencies, seeking access to the patient data.

In a further embodiment, patient data is safeguarded against unauthorized disclosure to third parties, including during collection, assembly, evaluation, transmission, and storage, to protect patient privacy and comply with recently enacted medical information privacy laws, such as the Health Insurance Portability and Accountability Act (HIPAA) and the European Privacy Directive. At a minimum, patient health information that identifies a particular individual with health-and medical-related information is treated as protectable, although other types of sensitive information in addition to or in lieu of specific patient health information could also be protectable. Additionally, for purposes of utilizing information in the population database 24 or third party data 26, comparison data can be de-identified, such that specific patient identification is not available.

Preferably, the server 13 is a server-grade computing platform configured as a uni-, multi-or distributed processing system, and the clients 23 are general-purpose computing workstations, such as a personal desktop or notebook computer. In addition, the patient management device 12, server 13 and clients 23 are programmable computing devices that respectively execute software programs and include components conventionally found in computing device, such as, for example, a central processing unit (CPU), memory, network interface, persistent storage, and various components for interconnecting these components.

Medical Device Hierarchy

A control strategy is a closed-loop combination of all actions taken by the various medical devices that affect the attainment of a therapy goal. A control strategy can involve a server 13, one or more patient management devices 12, and one or more medical devices 15-18. By default, the specificity of control, as delegated through the control strategy, is exercised over the operations performed by the server 13, patient management devices 12, and medical devices 15-18 to increase with immediacy to the patient 14. Conversely, the scope of feedback provided by these devices increases with distance from the patient 14. These characteristics can be formed into a hierarchy of bi-directional control and feedback. FIG. 2 is a tree diagram showing, by way of example, a control hierarchy 30 of medical devices for use in the patient management environment 10 of FIG. 1. The control hierarchy 30 is structured into four levels respectively corresponding to a server; one or more patient management devices; one or more medical devices that are each assigned to a patient management device; and sensors and effectors that respectively measure physiological data and deliver therapy.

A server is at the top or root level 31 of the control hierarchy 30 and serves as the primary controller for the patient management environment 10. From the prospective of the server, the successive levels 32-34 of the control hierarchy 30 constitute the physical plant over which control is exercised and from which feedback is received.

The patient management devices form an intermediate level 32 of the control hierarchy 30 and interface to both the server and one or more medical devices. In one embodiment, each patient management device is uniquely identified to a single patient 14. In a further environment, a patient management device can be shared by a plurality of patients 14. Each patient management device operates as a controller over the medical devices assigned, which constitute the physical plant over which the patient management device exercises control and from which feedback is received. In addition, each patient management device sends feedback to the server and receives control in the form of sub-goals from the server.

The medical devices form a penultimate level 33 of the control hierarchy 30 and can include sensors, effectors, or both, which are on the bottom or terminal level 34 of the control hierarchy 30. Each medical device functions as a controller over the sensors and effectors, which, in combination with the patient 14, constitute the physical plant over which control is exercised via the effectors and from which feedback is received from the sensors. In addition, each medical device sends feedback to the patient management device and receives control in the form of sub-goals from the patient management device.

In a strict control hierarchy 30, control is only exercised over and feedback is only received from the devices assigned to the next immediate level of the control hierarchy 30. In a more relaxed and pragmatic control hierarchy 30, control can flow down to and feedback can be received from the devices in any successive level of the control hierarchy 30, with one exception. Each medical device is physically coupled to sensors and effectors and operates under an event response control strategy, which does not admit to cooperative external control. However, a server can still exercise control over and receive feedback directly from patient medical devices and medical devices.

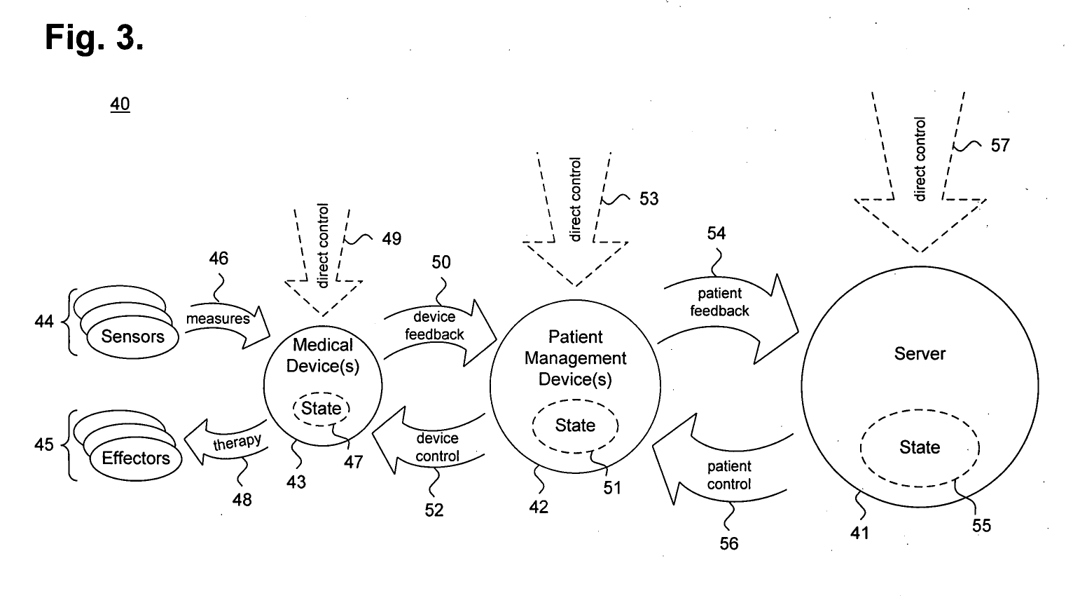

When executing as a closed loop control system, outside control and feedback reporting are not provided. However, direct control over the server, patient medical devices, and medical devices is possible and, for bootstrapping the server, necessary to specify initial therapy goals and implementing actions, including control parameters, environmental settings, and hierarchy assignments, such as further described below with reference to FIG. 3. Other levels or configurations and arrangements of tiered hierarchical control in addition to or lieu of a tree structure are also possible.

Data Flow

Generally, control flows downward in increasing levels of specificity and feedback flows upward in increasing levels of detail and temporal scope. FIG. 3 is a data flow diagram showing hierarchical medical device control and feedback 40 in the patient management environment 10 of FIG. 1. The server 41 is operatively coupled to a one or more patient management devices 42, which are each in turn operatively coupled to one or more medical devices 43. Each medical device 43 includes one or more sensors 44, one or more effectors 45, or a combination of both sensors 44 and effectors 45 depending upon the type of medical device.

The server 41 exercises patient-level control 56 over the patient management device 42 assigned and, in a further embodiment, device-level control 52 over medical devices 43. Each patient management device 42 exercises device-level control 52 over the medical devices 43 assigned. As event/response control devices, each medical device 43 maintains exclusive control over the interfaced sensors 44 and effectors 45. Each medical device 43 delivers therapy 48, such as pacing stimuli, through the interfaced effectors 45 and receives physiological measures 46 from the interfaced sensors 44. The received physiological measures 46 are transiently staged by the medical device 43 as limited state 47 before being uploaded to the assigned patient management device 42 and, in a further embodiment, the server 41, as device-level feedback 50 that can be analyzed with local state 51, 55 against the current control strategy. Each patient management device 42 uploads patient-level feedback 54 to the server 41, which can include physiological measures, parametric data, and environmental parameters as well as the results of local analyses and unprocessed device-level feedback 50. In addition to analyzing patient-level feedback 54 and, in a further embodiment, device-level feedback 50, the server 41 maintains access to the patient population database 24 and, in a further embodiment, third party data 26, which can both be factored into the analyses performed by the server 41. Other types of feedback and data access, exchange, and storage are possible.

A control strategy is implemented through goals and sub-goals that are delegated to devices in order of increasing specificity relative to the level of control exercised over the patient 14. Patient-level control 56, for instance, delegates a control strategy specific to a particular patient 14 who is uniquely assigned to a patient management device 42. Patient-level control 56 can affect one or more individual medical devices 43. Similarly, device-level control 52 delegates a control strategy specific to a particular medical device 43 based on the medical device type and the indicated form of therapy.

Direct control 49, 53, 57 can respectively be exercised over each medical device 43, patient management device 42, and the server 41, as would be necessary, for example, to set up the initial control parameters and environment settings necessary for each device to join into a hierarchical control strategy. Direct control over a medical device 43 can be provided through a programmer 22 (shown in FIG. 1) or patient management device 42. Direct control over a patient management device 42 could be provided through a client 23 (also shown in FIG. 1) or via a user interface of the patient management device 42. Direct control over the server 41 could be provided through a client 23.

Conversely, the detail and temporal scope of feedback increases as the available resources for storing and processing feedback increase and as the number of individual sources of feedback grow. Medical devices 43, as event/response control systems, maintain only limited state 47 in which to store temporarily physiological measures 47 received from interfaced sensors 44. With limited processing and power budget resources, each medical device 47 is typically constrained to limit processing of the physiological measures 46 to the extent necessary to determine the applicability to therapy delivery to the patient 14. Patient management devices 42 enjoy significantly more capable resources, including processing and storage, in which to store and analyze device-level feedback 50, which can be evaluated against the control strategy and analyzed, for instance, for trends indicating a progression, regression, absence, onset, or status quo of a physiological condition, and changes to the control strategy exercised by the assigned medical devices 43 can be effected through device control 52, which can include control parameters to reprogram assigned medical devices 43. The server 41 enjoys processing and storage resources at least on par with the patient management devices 42 and, typically, has far more capable resources. However, the wider range of sources of feedback, including patient-level feedback 54 from a plurality of patient management devices 42 and, in a further embodiment, directly-received device-level feedback 50 from a plurality of medical devices 43, introduce a richness of patient data at a population level that enables the server 41 to perform a wider range of comparative analyses across a spectrum of patient characteristics and health conditions, such as described in commonly-assigned U.S. patent application, entitled “System And Method For Providing Goal-Oriented Patient Management Based Upon Comparative Population Data Analysis,” Ser. No.______, filed on Jan. 19, 2006, pending, the disclosure of which is incorporated by reference. Other forms of analyses and processing are possible.

Data Set Examples

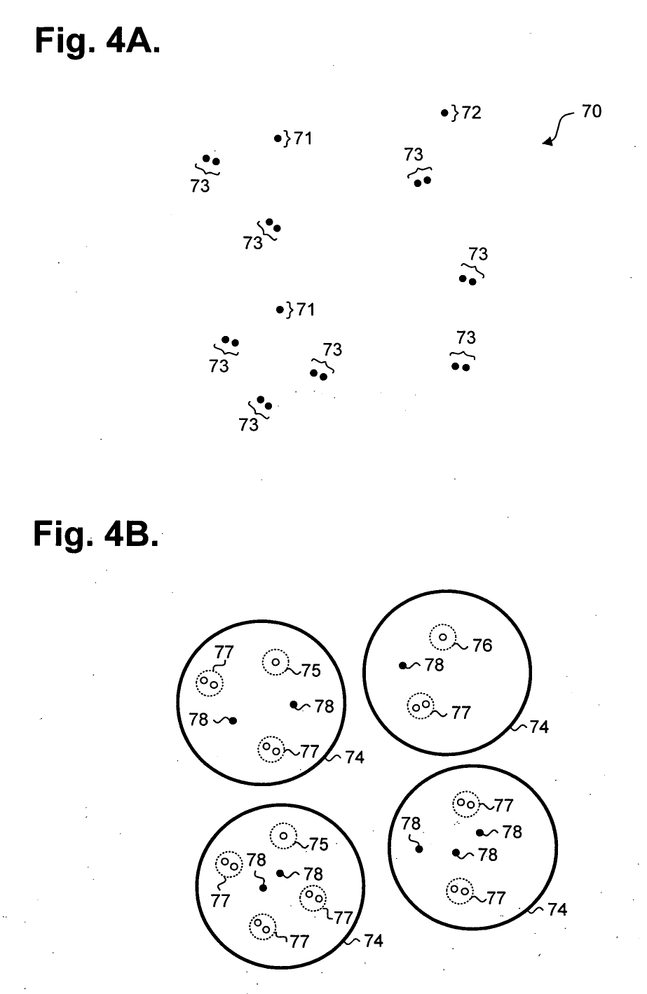

Each device maintains data sets that include feedback and control strategy data. FIGS. 4A-C are Venn diagrams showing, by way of example, data sets 70 as maintained in the patient management environment 10 of FIG. 1. The composition of each data set reflects the capabilities and storage capacities of the device. For instance, the data sets maintained by each medical device 43 are the most constrained due to the limited processing and storage resources available, whereas the data sets maintained by each patient management device 42 and by the server 41 are less constrained in terms of both processing and storage resources. Referring first to FIG. 4A, the data sets 70 maintained by medical devices 43 can include physiological measures or locally-generated data and analyses (“measures”) 71, control parameters 72, or a combination of measures and control parameters 73, depending upon the type of medical device.

Patient management devices 42 have greater processing capabilities and storage capacities than medical devices 43. Referring next to FIG. 4B, the data sets 74 maintained by patient management devices 42 can include measures recorded or generated by assigned medical devices 75, control parameters of assigned medical devices 76, or a combination of measures and control parameters 77. The measures 75 are provided as device-level feedback 50. In addition, the data sets 74 can also include physiological measures or data and analyses 78 that have respectively been locally measured or generated by the patient management devices 42.

Finally, the server 41 has a patient population-wide prospective, which potentially encompasses all of the individual data sets for assigned patient management device 42 and medical devices 43. Referring to FIG. 4C, the data set 79 maintained by the server 41 can include all of the medical device data sets and patient management device data sets. In addition, the data set 79 can also include data and analyses (not shown) that have been locally generated by the server 41. Other forms, combinations, and compositions of data sets are possible.

Sampling

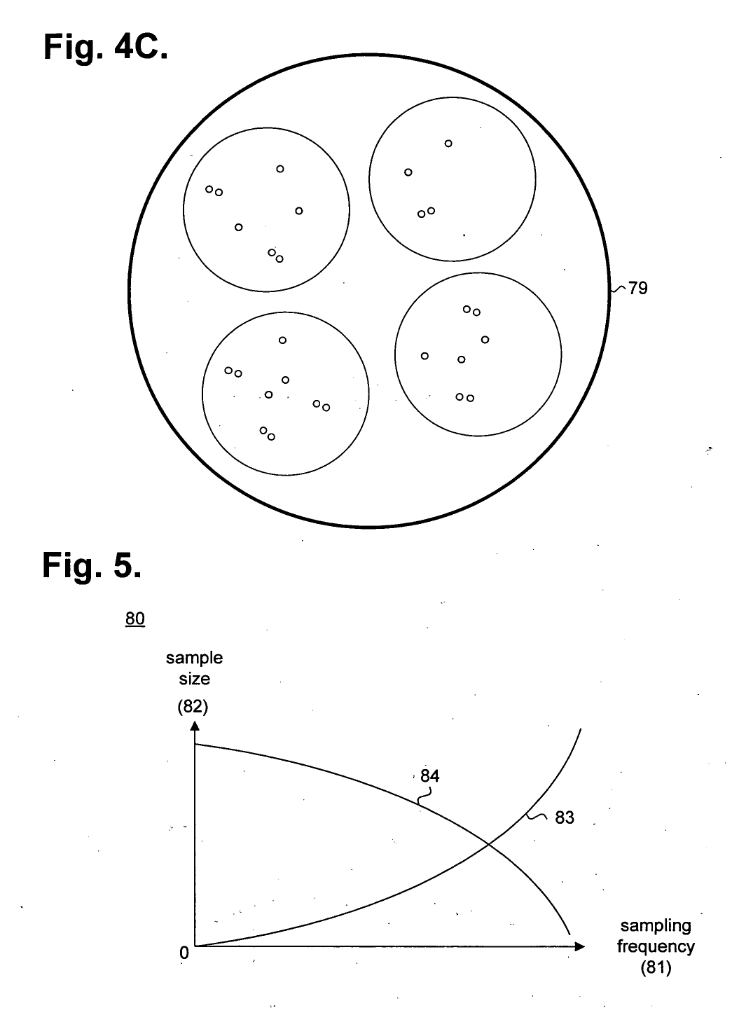

The detail and temporal scope of feedback grows as the number of independent sources and the rates of sampling applied at each hierarchy level increase. FIG. 5 is a graph 80 showing the relationship between sampling frequency and sample size. The x-axis represents the sampling frequency 81 and the y-axis represents the sample size 82.

In a control system with unlimited sampling resources, including state, an unconstrained sample size will continue to increase as a function 83 of the sampling frequency. However, due to the inherent limits in sampling resources in discrete devices, particularly with respect to medical devices 43 and the limited state 47 available for storing samples, the sample size instead decreases as a function 84 of the sampling frequency with the smallest samples being collected with the highest frequency by the medical devices 43 and largest samples being collected by the server 41 and patient management devices 42 with the lowest frequencies. Thus, medical devices 43 generally employ sampling rates in the millisecond range, while dedicated patient management devices 42 can sample on an hourly or daily basis and the server 41 can sample on a daily, weekly or monthly basis. The differences in sampling frequency allow each respective device to accumulate additional patient data samples and, where resources permit, to perform comparative analyses on the patient data to summarize and identify trends. Other sampling frequency and samples size relationships between the medical devices are possible.

Server Method

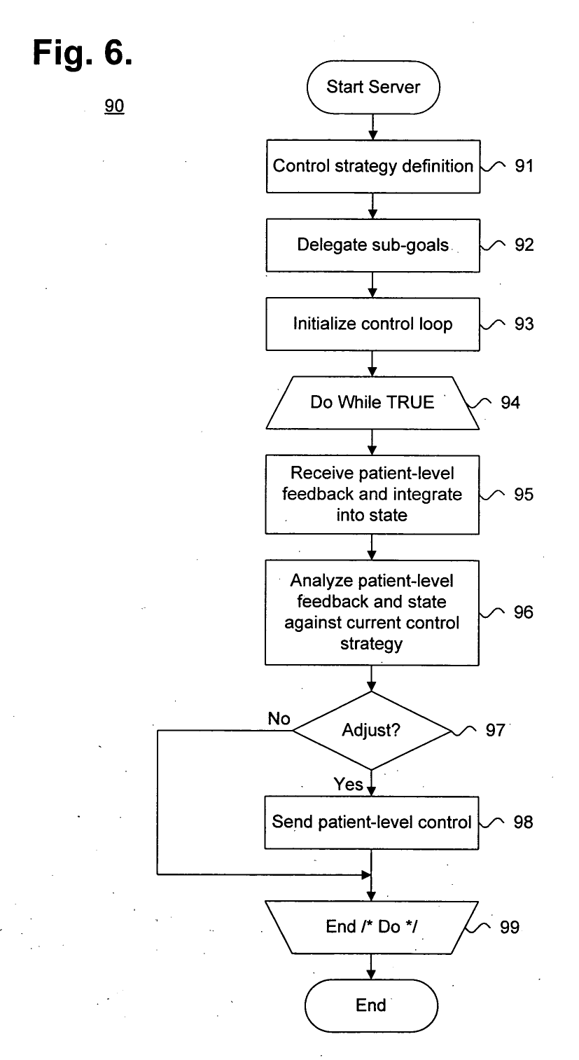

The operations performed by the server 41, patient management devices 42, and medical devices 43 are dependent upon the applicable sources and destinations of feedback and control strategy. Except when provided direct control 57 from external sources, such as a clinician providing instructions through a client 23, the server 41 functions as an autonomous closed loop controller that exercises control over the patient management devices 42 and medical devices 43 assigned to the control hierarchy. FIG. 6 is a flow diagram showing a method 90 for providing hierarchical medical device control for automated patient management for use on the server 41 of FIG. 3. The method 90 is generally performed on the server 41, but could also be performed on a patient management device 42 or client 23 with sufficient resources and interconnections.

Initially, a control strategy is defined (block 91), which can be provided through direct control 57 or by analysis of the patient population database 24 or other sources, such as described in commonly-assigned U.S. patent application, entitled “System And Method For Providing Goal-Oriented Patient Management Based Upon Comparative Population Data Analysis,” Ser. No.______, filed on Jan. 19, 2006, pending, the disclosure of which is incorporated by reference. The control strategy can be decomposed into sub-goals that can be delegated to patient management devices 42 as patient-level control 56 and, in a further embodiment, as device-level control 52 to medical devices 43 (block 92). A closed control loop is then initialized (block 93) by verifying, as necessary, connectivity to each assigned patient management device 42 and medical device 43 and confirming satisfactory operational statuses. The continuous closed control loop (blocks 94-99) is then performed until the processing infrastructure, for instance, the server 41, terminates execution.

During each cycle (block 94), patient-level feedback 54 is received and integrated into the state 55 maintained by the server 41(block 95). The patient-level feedback 54 and state 55 are analyzed against the current control strategy, such as through data mining (block 96). In one embodiment, the state is represented as a matrix dimensioned temporally as a set of vectors for the tracked patient data, including physiological measures, control parameters, and environmental parameters. Other types of tracked patient data and forms of internal state representation are also possible. If based on the analysis, the control strategy requires adjustment (block 97), revised patient-level control 56 and, in a further embodiment, device-level control 52, are sent to the appropriate device (block 98). Closed control loop processing (block 99) is performed continually, but can be subject to interruption or modification by external sources, such as direct control 57.

Patient Management Device Method

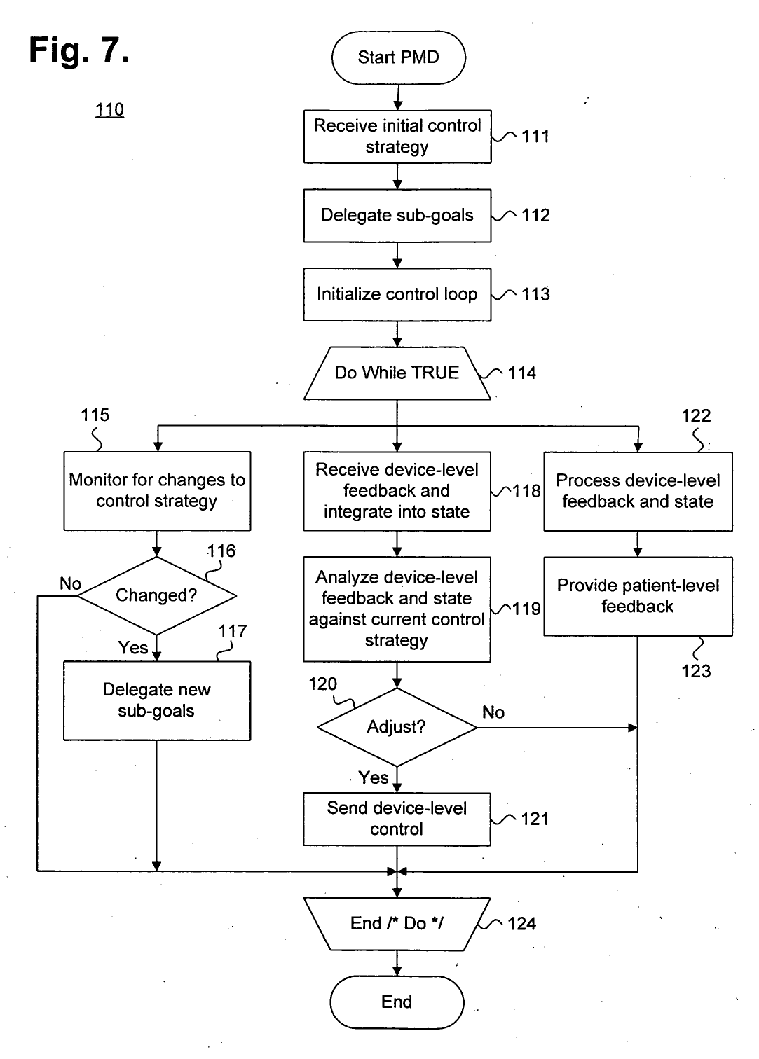

Except when provided direct control 53, each patient management device 42 also functions as an autonomous closed loop controller that exercises control over the medical devices 43 assigned to the control hierarchy, subject to patient-level control 56 delegated by the server 41. FIG. 7 is a flow diagram showing a method 110 for providing hierarchical medical device control for automated patient management for use on a patient management device 42 of FIG. 3. The method 110 is preferably performed by a patient management device 42 but, in a further embodiment, could also be performed by a server 41 or client 23.

Each patient management device 42 operates in two logical roles. First, as part of the physical plant of the control system implemented by the server 41, each patient management device 42 receives patient-level control 56, which defines the initial control strategy to be executed (block 111). Second, as a controller to the medical devices 43 assigned, each patient management device 42 delegates sub-goals (block 112), which, in a further embodiment, could also be sent to other patient management devices 42 or the server 41. Other patient management device functions are possible. A closed control loop is then initialized (block 113) by verifying, as necessary, connectivity to each assigned medical device 43 and confirming satisfactory operational statuses. The continuous closed control loop (blocks 114-124) is then performed until the processing infrastructure, for instance, the patient management device 42, terminates execution.

During each cycle (block 114), three threads of control are performed to receive patient-level control 56 (blocks 115-117), receive device-level feedback 50 and send device control 52 (blocks 118-121), and send patient-level feedback 54 (blocks 122-123). In the patient-level control thread, changes to the control strategy that are received as patient-level control 56 from the server 41 are monitored (block 115). If the control strategy has changed (block 116), the control parameters for the patient management device 42, and if applicable, for one or more of the attached medical devices 43, are revised (block 117). Device-level control 52 to the appropriate assigned medical devices 43 is also sent. In the medical device control thread, patient data is received as device-level feedback 50 from each assigned medical device 43 and is integrated into the state 51 for the patient management device 42 (block 118). The device-level feedback 50 and state 51 are analyzed against the current control strategy (block 119) and, if the current control strategy requires adjustment (block 120), device-level control 52 is sent to the appropriate assigned medical devices 43 (block 121). Finally, in the patient-level feedback thread, device-level feedback 50 and state 51 are processed (block 122) and provided as patient-level feedback 54 to the server 41 (block 123). Processing can include summarizing and extrapolating the patient data over those devices that constitute the physical plant of the patient management device 42. Other types of processing are possible. Closed control loop processing (block 124) is performed continually, but can be subject to interruption or modification by external sources, such as direct control 53.

Medical Device Method

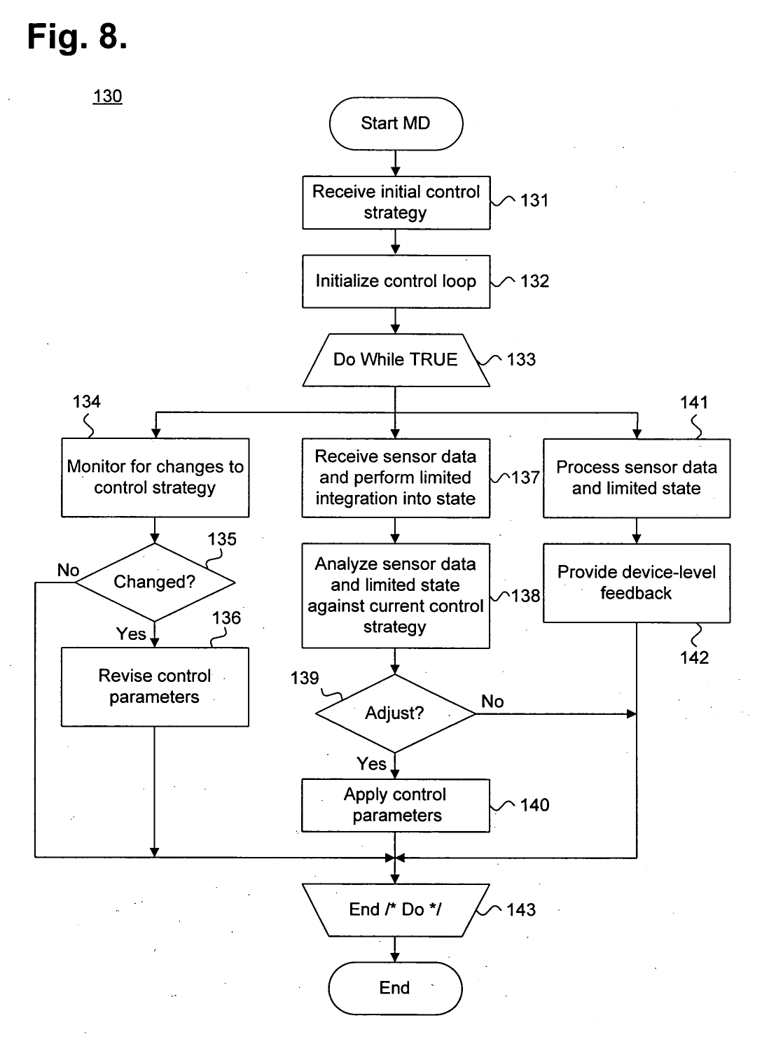

Except when provided direct control 49, each medical device 43 also functions as an autonomous event/response controller that receives physiological measures 46 from sensors 44 and delivers therapy 48 through effectors 45, subject to patient-level control 56 delegated by an associated patient management device 42 and, in a further embodiment, the server 41. FIG. 8 is a flow diagram showing a method 130 for providing hierarchical medical device control for automated patient management for use on a medical device 43 of FIG. 3. The method 130 is performed by a medical device 43.

Each medical device 43 operates in two logical roles. First, as part of the physical plant of the control system implemented by the associated patient management device 42 and, in a further embodiment, the server 41, each medical device 43 receives device-level control 52, which defines the initial control strategy to be executed (block 131). Second, as an event/response controller, each medical device 43 delivers therapy, such as pacing stimuli, and receives physiological measures. Other medical device functions are possible. An event/response control loop is then initialized (block 132) by confirming satisfactory operational statuses of the sensors 44 and effectors 45. The event/response control loop (blocks 133-143) is then performed until the processing infrastructure, for instance, the medical device 43, terminates execution.

During each cycle (block 133), three threads of control are performed to receive device-level control 52 (blocks 134-136), control sensors 44 and effectors 45 (blocks 137-140), and send device-level feedback 50 (blocks 141-142). In the device-level control thread, changes to the control strategy that are received as device-level control 52 from the assigned patient management device 42 and, in a further embodiment, the server 41 are monitored (block 134). If the control strategy has changed (block 135), the control parameters for the medical device 43 are revised (block 136), which could affect the event occurrence and response characteristics of the medical device 43. In the sensors 44 and effectors 45, control thread, physiological measures 46 are received from each sensor 44 and are integrated into the limited state 47 for the medical device 43 (block 137). The physiological measures 46 and limited state 47 are analyzed against the current control strategy (block 138) and, if the current control strategy requires adjustment (block 139), revised control is applied to the interfaced sensors 44 and effectors 45 (block 140). Finally, in the device-level feedback thread, physiological measures 46 and the limited state 47 are processed (block 141) and provided as device-level feedback 50 to the associated patient management device 43 and, in a further embodiment, to the server 41 (block 142). Processing can include averaging or summarizing the physiological measures 46. Other types of processing are possible. Closed control loop processing (block 143) is performed continually, but can be subject to interruption or modification by external sources, such as direct control 49.

Server Structure

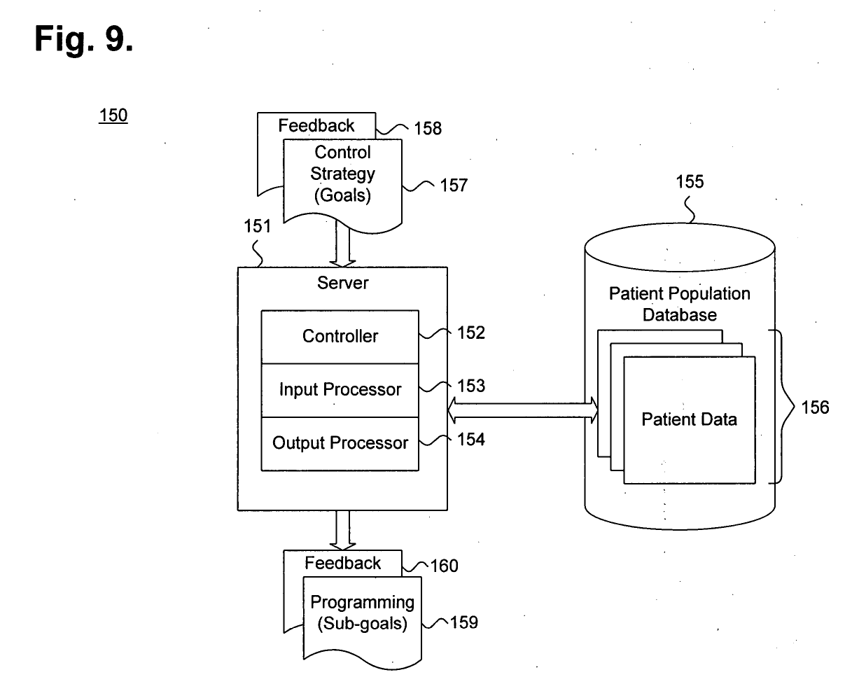

Generally, the server is responsible for exercising control over the patient management devices and medical devices assigned. FIG. 9 is a block diagram showing a system 150 for providing hierarchical medical device control for automated patient management for use on the server of FIG. 3. The server 151 implements the system 150 and executes a sequence of programmed process steps, such as described above with reference to FIG. 6, implemented, for instance, on a programmed digital computer system.

The server 151 includes a controller 152, input processor 153, and output processor 154. The server 151 also maintains an interface to the patient population database 155. The patient population database 155 is used to maintain patient data 156, which can include patient characteristics, wellness, treatment plans, regimens, and other types of information. The patient information 156 is maintained for those patients belonging to the population of patients managed by the server 151 as well as for other patients not strictly within the immediate patient population, such as retrieved from third party data sources 26.

The controller 152 processes feedback 158 that can include patient-level feedback 54 and, in a further embodiment, device-level feedback 50, and the patient data 156, which constitutes part of the state 55 maintained by the server 151. The state is analyzed against the current control strategy 157 to determine if changes to the current control strategy 157 are needed. Other types of analyses are possible. The feedback 158 and control strategy 157 are received by the input processor 153, which integrates the feedback 158 into the patient data 156 stored in the patient population database 155 and provides the control strategy 157 to the controller 152, which delegates programming 159 as sub-goals to assigned patient management devices 42, and, in a further embodiment, medical devices 43. The output processor 154 sends programming 159 and can also provide feedback 160 to external sources, such as clients 23 (shown in FIG. 1) or displays associated with the patient management device for further display and analysis. Other components and functionality are possible.

Patient Management Device Structure

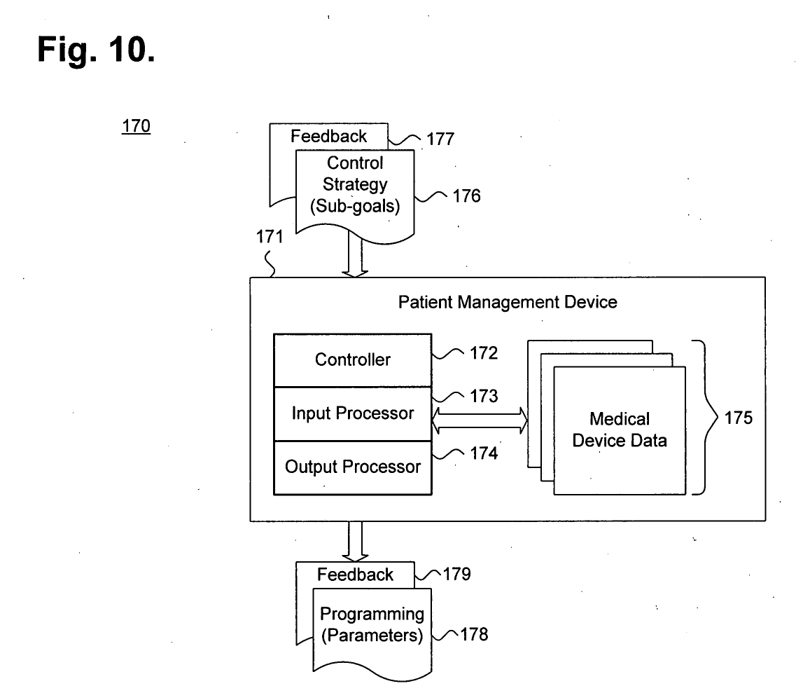

Generally, each patient management device is responsible for exercising control over the medical devices assigned. FIG. 10 is a block diagram showing a system 170 for providing hierarchical medical device control for automated patient management for use on a patient management device of FIG. 3. The patient management device 171 implements the system 170 and executes a sequence of programmed process steps, such as described above with reference to FIG. 7, implementing, for instance, on a programmed digital computer system.

The patient management device 171 includes a controller 172, input processor 173, and output processor 174. Medical device data 175 is maintained for the assigned medical devices 43. The controller 172 processes feedback 177 that can include device-level feedback 50, which constitutes part of the state 51 maintained by the patient management device 42. The state is analyzed against the current control strategy 176 to determine if changes to the current control strategy 176 are needed. Other types of analyses are possible; The feedback 177 and control strategy 176 are received by the input processor 173, which integrates the feedback 177 into the medical device data 175 and provides the control strategy 176 to the controller 172, which delegates programming 178 as control parameters to assigned medical devices 43. The output processor 174 sends programming 178 and can also provide feedback 179 to the server 41 and external sources, such as clients 23 (shown in FIG. 1) for further display and analysis. Other components and functionality are possible.

Medical Device Structure

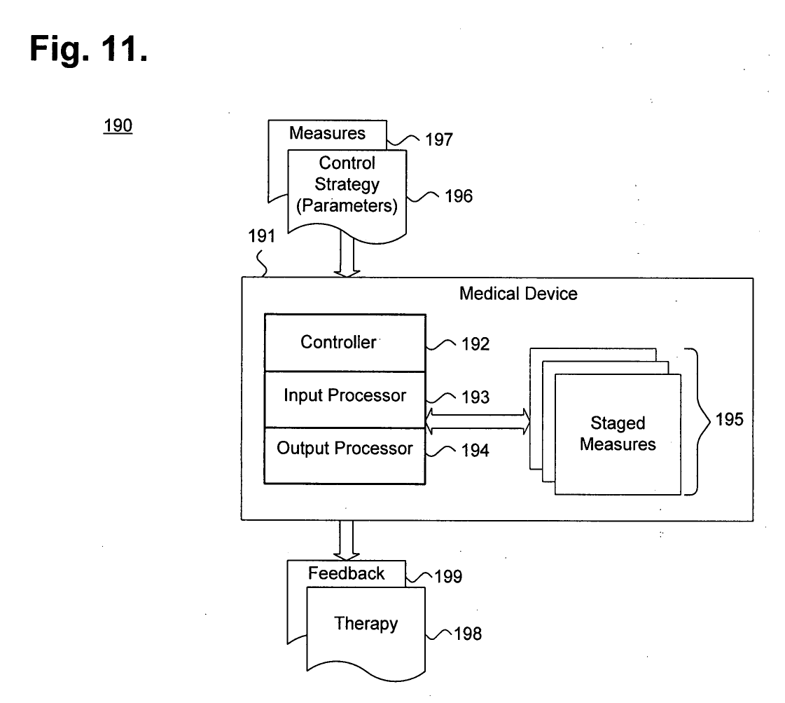

Generally, each medical device is responsible for monitoring physiological measures and providing therapy to the patient 14. FIG. 11 is a block diagram showing a system 190 for providing hierarchical medical device control for automated patient management for use on a medical device of FIG. 3. The medical device 191 implements the system 190 and executes a sequence of programmed process steps, such as described above with reference to FIG. 8, implemented, for instance, on an embedded microprocessor-based system.

The medical device 191 includes a controller 192, input processor 193, and output processor 194. Staged measures 195 are maintained for the sensors 44. The controller 192 processes physiological measures 197, which constitute part of the limited state 47 maintained by each medical device 190. The limited state 47 is analyzed against the current control strategy 196 to determine if changes to the programming are required. Other types of analyses are possible. The physiological measures 197 and control strategy 196 are received by the input processor 193, which integrates the physiological measures 197 into the staged measures 195 and provides the control strategy 196 to the controller 192 as programming that is implemented to change the event occurrence and response control performed by the medical device 190. The output processor 194 delivers therapy 198 to the patient 14 and can also provide feedback 199 to the associated patient management device 42 and, in a further embodiment, the server 41, plus external sources, such as clients 23 (shown in FIG. 1) for further display and analysis. Other components and functionality are possible.

While the invention has been particularly shown and described as referenced to the embodiments thereof, those skilled in the art will understand that the foregoing and other changes in form and detail may be made therein without departing from the spirit and scope of the invention.

What is claimed is:

1. A system for providing hierarchical medical device control for automated patient management, comprising:
a plurality of medical devices operatively coupled to a processor on a substantially continual basis to receive sensor data;
a control strategy assigned to the processor to specify actions to be taken by the medical devices to affect the attainment of a therapy goal; and
stored state selected from the group comprising a history of changes to the control strategy and past sensor data received from the medical devices, wherein the processor further comprises:
an input processor to periodically receive feedback comprising new sensor data from the medical devices;
a controller to analyze the feedback and the state against the actions specified in the control strategy; and
an output processor to provide control to one or more medical device in response to an actionable change from the actions specified in the control strategy.

2. A system according to claim 1, further comprising:
a control hierarchy to implement the control strategy structured into a plurality of logical levels of control, each logical control level comprising devices comprising at least one of a medical device and a processor.

3. A system according to claim 2, wherein the devices are selected for each logical control level based on feedback considerations selected from the group comprising sampling frequency, sample size, and sample composition.

4. A system according to claim 1, further comprising.
vectorized parameters to express the control strategy, wherein the actionable change is represented as measurable deviation from the control strategy.

5. A system according to claim 1, wherein the state is expressed as at least one of temporal and dimensional data.

6. A system according to claim 1, further comprising:
a threshold to specify the actionable change between at least one of the feedback and the state versus the control strategy.

7. A system according to claim 1, wherein a status of the processor is forwarded in response to an actionable change from the control strategy to a centralized processor operatively coupled on a regular basis.

8. A system according to claim 1, wherein instructions affecting the control strategy are received from a centralized processor operatively coupled on a regular basis.

9. A system according to claim 1, wherein the processor is selected from the group comprising a patient management device and a centralized server.

10. A system according to claim 1, wherein the medical device is selected from the group comprising an external medical device, an internal medical device, an external medical sensor, and an internal medical sensor.

11. A system according to claim 1, wherein the feedback comprises at least one of physiological measures, parametric data, and environmental parameters.

12. A method for providing hierarchical medical device control for automated patient management, comprising:
operatively coupling a processor to a plurality of medical devices on a substantially continual basis to receive sensor data;
assigning a control strategy to the processor to specify actions to be taken by the medical devices to affect the attainment of a therapy goal;
maintaining state selected from the group comprising a history of changes to the control strategy and past sensor data received from the medical devices;
periodically receiving feedback comprising new sensor data from the medical devices and analyzing the feedback and the state against the actions specified in the control strategy; and
providing control to one or more medical device in response to an actionable change from the actions specified in the control strategy.

13. A method according to claim 12, further comprising:
implementing the control strategy by structuring a control hierarchy comprising plurality of logical levels of control, each logical control level comprising devices comprising at least one of a medical device and a processor.

14. A method according to claim 13, further comprising:
selecting the devices for each logical control level based on feedback considerations selected from the group comprising sampling frequency, sample size, and sample composition.

15. A method according to claim 12, further comprising:
expressing the control strategy as vectorized parameters, wherein the actionable change is represented as measurable deviation from the control strategy.

16. A method according to claim 12, further comprising:
expressing the state as at least one of temporal and dimensional data.

17. A method according to claim 12, further comprising:
specifying the actionable change as a threshold between at least one of the feedback and the state versus the control strategy.

18. A method according to claim 12, further comprising:
forwarding a status of the processor in response to an actionable change from the control strategy to a centralized processor operatively coupled on a regular basis.

19. A method according to claim 12, further comprising:
receiving instructions affecting the control strategy from a centralized processor operatively coupled on a regular basis.

20. A method according to claim 12, wherein the processor is selected from the group comprising a patient management device and a centralized server.

21. A method according to claim 12, wherein the medical device is selected from the group comprising an external medical device, an internal medical device, an external medical sensor, and an internal medical sensor.

22. A method according to claim 12, wherein the feedback comprises at least one of physiological measures, parametric data, and environmental parameters.

23. A computer-readable storage medium holding code for performing the method according to claim 12.

24. An apparatus for providing hierarchical medical device control for automated patient management, comprising:
means for operatively coupling a processor to a plurality of medical devices on a substantially continual basis to receive sensor data;
means for assigning a control strategy to the processor to specify actions to be taken by the medical devices to affect the attainment of a therapy goal;
means for maintaining state selected from the group comprising a history of changes to the control strategy and past sensor data received from the medical devices;
means for periodically receiving feedback comprising new sensor data from the medical devices and means for analyzing the feedback and the state against the actions specified in the control strategy; and
means for providing control to one or more medical device in response to an actionable change from the actions specified in the control strategy.
